(19) United States
(12) Patent Application Publication
(10) Pub. No.: US 2024/0009424 A1
(43) Pub. Date: Jan. 11, 2024

(54) EXTENDABLE LUMEN CATHETER DEVICE

(71) Applicant: SCIENTIA VASCULAR, INC., West Valley City, UT (US)

(72) Inventors: John A. Lippert, Park City, UT (US); Edward J. Snyder, Park City, UT (US)

(21) Appl. No.: 18/218,974

(22) Filed: Jul. 6, 2023

Related U.S. Application Data
(60) Provisional application No. 63/359,690, filed on Jul. 8, 2022.

(51) Int. Cl. A61M 25/01; A61M 25/00

(52) U.S. Cl. CPC A61M 25/0102; A61M 25/0026; A61M 25/005; A61M 25/0108; A61M 25/0045

(57) ABSTRACT

Disclosed is a catheter system including an outer member, an inner member that translates within a lumen of the outer member, and a stylette configured to fit within a lumen of the inner member. The inner member is translatable within the outer member to selectively extend beyond a distal end of the outer member, and the stylette has greater bending stiffness and/or columnar stiffness than the inner member to assist in navigating the inner member to targeted anatomy. This catheter system can get close to targeted anatomy to allow for better treatment. In particular, the catheter system is useful in adhesive embolization.

### CROSS-REFERENCE TO RELATED APPLICATIONS

This application claims the benefit of and priority to U.S. Provisional Patent Application No. 63/359,690, filed Jul. 8, 2022, and titled “Extendable Lumen Catheter Device,” which is incorporated herein in its entirety by this reference.

### BACKGROUND

Catheters and guidewire devices are commonly used to target specific medical issues in a patient's body. Lesions (e.g., vascular lesions) often require catheters to be moved along narrow blood vessels to reach and embolize the lesions. Typically, adhesive embolization is performed by guiding a microcatheter to the treatment site and then using an adhesive (interchangeably referred to herein as an embolic agent and/or embolic adhesive), typically a cyanoacrylate (e.g., n-BCA) or the product sold under the tradename Onyx (which includes ethylene vinyl-alcohol copolymer (EVOH), dimethyl-sulfoxide (DMSO), and micronized tantalum powder (TA)) to embolize the lesion. For example, this procedure is common when treating lesions in the Middle Meningeal Artery (MMA) during an MMA embolism.

One of the challenges of moving the catheter to the treatment site is the size and pathway of the blood vessels. The blood vessels are often narrow and tortuous, making it difficult to get to the targeted anatomy at issue. Catheters and guidewires sometimes are unable to get to the targeted location or cannot get as close to it as desired. Often, the practitioner must inject the adhesive from a greater than preferred distance to treat the lesion(s).

For example, instead of applying adhesive directly to the embolization site, the practitioner may be required to release adhesive from a more distant point. This requires filling the blood vessel(s) with adhesive until it reaches the target site. This also complicates the already difficult balancing act between getting the adhesive to the target before it polymerizes but also allowing it to polymerize before passing too far down the blood vessel past the target. Also, the additional space within the vessel(s), that otherwise would not have been filled with adhesive had the catheter progressed closer to the target, unnecessarily loses its physiological function. Moreover, the adhesive can be quite expensive, and the additional amount required just to fill the space between the catheter and the target can significantly add to the overall cost of the procedure.

In sum, current medical techniques requiring catheters have multiple variables that can affect the procedure. Some of these variables include the vascular path to reach the targeted anatomy, how the catheter is able to move through that pathway, the type of adhesive used and its properties, and other medical complications that could arise. Accordingly, there is a long felt and ongoing need for improved catheters that minimize the number of variables during medical procedures and are able to move easily throughout the vascular system.

### SUMMARY

The present disclosure relates to a catheter system which includes an outer member and an inner member. Embodiments described herein solve one or more problems related to adhesive embolization that requires the use of a catheter. During adhesive embolization procedures, the closer the catheter can get to the lesion or treatment site, the better the practitioner will be able to manage variables associated with the procedure and the less adhesive will be necessary. The catheter system described herein uses an outer member to initially navigate towards the targeted anatomy and then inserts a second inner member into the outer member and distally past the distal end of the outer member to move even closer to the targeted treatment site.

Embodiments of the catheter system include a stylette which can be inserted into the inner member. This allows for increased rigidity which may aid in navigating the inner member towards the targeted anatomy. That is, the stylette may be configured to have greater bending stiffness and/or columnar stiffness than the inner member. This stylette may include a radiopaque marker, which when inserted can show the position of the stylette and thus the inner member. Some embodiments may include a hydrophilic coating around the stylette, allowing the stylette to slide into the inner member more easily.

In some embodiments, the outer member and stylette may be microfabricated to provide properties such as flexibility when navigating through the vascular system. The outer member and stylette may be fabricated to create circumferentially extending rings and axially extending beams by various cuts of similar of varying depths, widths, and spacing, which may occur with or without angular offsets between successive beams.

Other embodiments may include an inner member where the distal section is selectively detachable. The distal section may detach after injection of an embolic agent. For example, if the distal section of the inner member bonds to the adhesive, the practitioner can beneficially detach the distal section so that the remainder of the inner member and the rest of the catheter system can be safely removed from the patient.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an example of an outer member with a lumen and a hub;

FIG. 2 illustrates an example of an inner member with a lumen, selectively detachable distal section, and a hub;

FIG. 3 illustrates an example of a stylette with a microfabricated distal section, a radiopaque marker at a distal end, and a connector element;

FIG. 4 illustrates an example of a catheter system including the outer member, inner member, and stylette, where the inner member has a selectively detachable distal section; and

FIG. 5 illustrates an example of the catheter system including the outer member and the inner member where the distal section of the inner member has been detached.

### DETAILED DESCRIPTION

### Example Embodiments

The following detailed description solves one or more of the problems discussed above. As explained in greater detail below, the outer member can be moved towards the targeted anatomy until it cannot readily go any farther. At that point, the inner member may be inserted into the outer member and translated through the outer member until the distal end of the inner member moves distally past the distal end of the outer member, thereby moving the functional distal end of catheter system closer to the targeted anatomy. The closer the catheter system is to the targeted anatomy, the better the practitioner can employ the desired treatment.

For example, when injecting an adhesive, the closer the distal end of the system is to the target anatomy, the less adhesive is necessary to seal the targeted lesion. The adhesive is relatively expensive, and injecting adhesive into the vessels adds variability to the overall procedure. Thus, the ability to navigate the functional distal end of the catheter system closer to the target anatomy (or at least get closer than where conventional systems are used) can beneficially reduce procedure variability and costs, leading to improved patient outcomes.

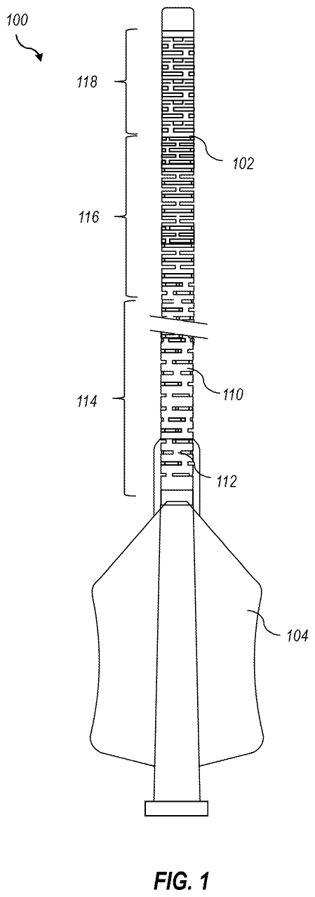

FIG. 1 illustrates an example of an outer member 100 that includes a hub 104 and an elongated member 102. The hub 104 may be coupled to the elongated member 102 using adhesive, a friction fit, molding, and/or other appropriate attachment means. The elongated member 102 in the illustrated embodiment is formed from nitinol. Other embodiments may use other materials for the elongated member 102. For example, the elongated member 102, may be formed of one or more polymers, such as polyether ether ketone (PEEK) or polyaryl ether ketone (PAEK).

The elongated member 102 is microfabricated to include circumferentially extending rings 110 and axially extending beams 112. The cut depth, width, and spacing may be consistent or may vary. Embodiments may include one-beam patterns or multiple-beam patterns where the beams are configured in a variety of arrangements. A one beam pattern includes one axially extending member between each successive circumferentially extending ring. Similarly, a two beam pattern includes two axially extending members between each successive circumferentially extending ring. Three beam and more than three beam patterns can be created in a similar fashion. In multiple beam patterns, the beams may be equally or randomly spaced. For example, in a two beam pattern, the two beams may be spaced 180 degrees from one another. In another embodiment, the two beams may be spaced 90 degrees from one another. Various additional cut patterns that may be utilized to form different configurations of rings and beams are disclosed in United States Patent Publication No. 2020/0121308, which is incorporated herein by this reference.

Angular offset arrangements can vary based on the amount of angular offset and/or how often an angular offset is applied between successive beams. Angular offset is the angle between successive beams. By rotating the position of successive beams, preferred bending orientations can be achieved or avoided. For example, a one beam pattern may have a 0 degree offset between successive beams (i.e., no angular offset). This angular offset would align all the beams along a “spine” of the outer member, and would result in preferred bending orientation towards the side where the beams are located (e.g., if the beams are located on a “right” side, the catheter will prefer to bend to the right side). Another example is a one beam pattern with a 180 degree offset between successive beams. A 180 degree angular offset reduces some of the preferred bending seen in the 0 degree offset example. Other embodiments may include other angular offsets therebetween. For example, a two beam pattern may have a 90 degree angular offset while a three beam pattern may have a 60 degree angular offset. As mentioned above, various additional cut patterns that may be utilized to form different configurations of rings and beams are disclosed in United States Patent Publication No. 2020/0121308, which is incorporated herein by this reference.

The embodiment shown in FIG. 1 includes three microfabricated sections: a first section 114, a second section 116, and a third section 118. In other embodiments, the number of microfabricated sections can include one section, two sections, three sections, or more than three sections. These microfabricated sections provide flexibility when bending the catheter through turns found in the vascular system while maintaining pushability to ensure the catheter can sufficiently move through the vessels.

The length, diameter, and stiffness of the outer member 100 may vary based on application needs. For example, the outer member's elongated member 102 may be about 50 cm to about 200 cm, though shorter or longer lengths may be utilized when appropriate. The diameter of the outer member's lumen may range from about 0.010 inches to 0.100 inches. Possible examples include lumen diameters of 0.013 inches, 0.017 inches, 0.021 inches, 0.027 inches, 0.030 inches, 0.035 inches, 0.038 inches, 0.045 inches, 0.065 inches, 0.086 inches, or a range including any two of the foregoing values as endpoints. The outer catheter 100 is configured to have a transitioning stiffness profile where the proximal section has a higher stiffness and less bending flexibility than the distal section, which has a lower stiffness and higher bending flexibility. The added flexibility in the distal section allows for easier navigation through tortuous vasculature, whereas more proximal sections beneficially provide greater pushability and torquability.

The stiffness profile gradient described above can be created by adjusting the microfabricated elongated member 102 and/or by utilizing different polymer materials. For example, the first microfabricated section 114 may be formed from a first polymer material, the second microfabricated section 116 may be formed from a second polymer material, and the third microfabricated section 118 may be formed of a third polymer material. The hardness and/or modulus of the first polymer material may be greater than the second polymer material, which may be greater than the third polymer material. The differences in polymer hardness and/or modulus thus affects the stiffness of the appropriate sections in the elongated member 102. The microfabricated pattern may also affect the overall stiffness of the elongated member 102. For example, a one beam pattern may have more flexibility than a three beam pattern. The gaps and/or cuts in the microfabricated portions may be filled with another polymer. For example, a soft polymer that allows flexibility (e.g., PEBAX) may fill in the gaps and/or cuts between the rings 110 and beams 112.

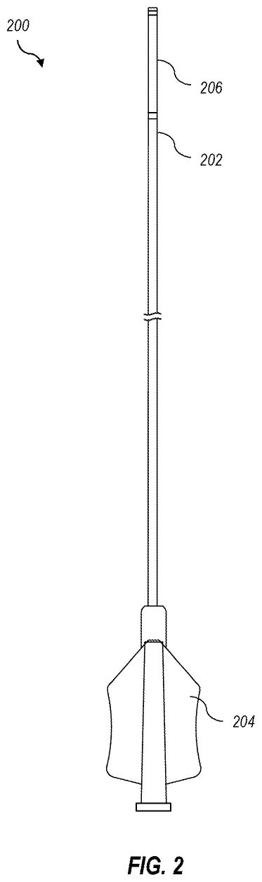

FIG. 2 illustrates an example of an inner member 200 including a hub 204 and an elongated member 202. An example of a hub 204 may be a Luer connector. The elongated member 202 is typically formed from a polymer. In some embodiments, one or more materials may be used which do not bond to an embolic adhesive that has been passed through the lumen of the elongated member 202. In some embodiments, the elongated member 202 includes a thermoplastic polyurethane (TPU), thermoplastic polyolefin (TPO), polyether block amide (PEBA), other suitable polymers, or combinations thereof.

The diameter of the elongated member 202 is preferably less than the diameter of the outer member's lumen to allow the inner member 200 to pass through the outer member's lumen. The inner member 200 is shown as having a detachable distal section 206. The joint between the detachable distal section 206 and the more proximal sections of the inner member 200 may include a perforation, groove, thinned section, partial break, intentional weak spot, or other mechanically weakened structure configured to detach the distal section 206 from the lumen 202 when sufficient tension is applied. In other embodiments, the lumen 202 may be continuous and not include a detachable distal section 206.

The length of the elongated member 202 is preferably long enough to pass entirely through and beyond the outer member's lumen. The length of the elongated member 202 may extend up to 40 cm beyond the outer catheter's lumen. For example, the elongated member 202 may have a length that allows extension of up to about 40, 35, 32, 18, 15, 10, 8, 5, or 2 cm beyond the outer member's distal end. The detachable distal section 206 similarly may vary in length. The detachable distal section 206 may be the last 2 cm of the elongated member 202. Other embodiments may include a detachable distal section 206 that is about 1.8, 1.5, 1.3, 1, 0.7, or 0.5 cm in length (or a length within a range with endpoints defined by any two of the foregoing values), for example.

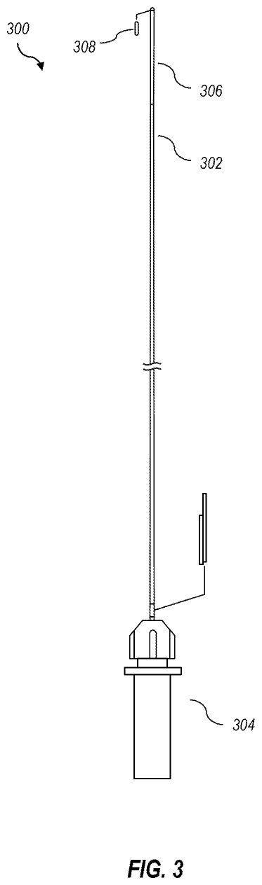

FIG. 3 illustrates an example of a stylette 300 including a connecting element 304 and an extending member 302. The extending member 302 is made of nitinol. Other embodiments may additionally or alternatively include other materials appropriate to be used in catheter stylettes, such as stainless steel or polymer/polymer blend materials (including those polymer materials described herein for other components of the system). The illustrated stylette 300 includes a microfabricated distal section 306. The microfabricated distal section 306 can include cuts at different depths, widths, and spacing to create circumferential rings and axial beams, similar to the outer member lumen 102 in FIG. 1. The distal section 306 of the stylette 300 is therefore less stiff and more flexible than more proximal sections. The microfabricated distal section 306 is preferably flexible enough to be atraumatic when pushed through the vasculature.

In another embodiment, the stylette 300 may omit a microfabricated distal section 306. In some embodiments, disposed at or near the distal end of the stylette 300 is a radiopaque marker 308 configured to align near or at the distal end of the inner member 200 to assist in visualization of the distal end of the inner member 200. Another embodiment omits the radiopaque marker 308. In some embodiments, the stylette 300 may include a hydrophilic coating to allow the stylette 300 to be inserted into and move through the inner member 200 more easily.

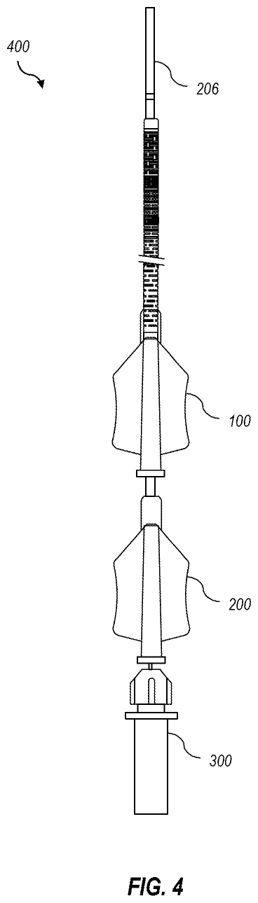

FIG. 4 illustrates an example of the fully assembled catheter system 400 including the outer member 100, inner member 200, and stylette 300. In an exemplary use, the outer member 100 will first be inserted into the vascular system and navigated towards the treatment site at the targeted anatomy. At some point, due to the tortuosity of the vasculature system, the outer member 100 may no longer be able to get closer to the targeted treatment site. In a conventional procedure, the practitioner would have to either apply the adhesive from a suboptimal distance or would have to retract the catheter and re-attempt navigation to get closer to the target. In contrast, when using the present catheter system 400, the inner member 200 is inserted into the outer member 100 and past the distal end of the outer member 100 to functionally extend the length of the system 400 and allow the distal end of the inner member 200 to get closer to the treatment site.

In some embodiments, multiple inner members 200 may be used with a single outer member 100. For example, a first inner member may be used to deliver an embolic agent to a targeted treatment site. The first inner member may then be removed from the system, and a second inner member may then be routed through the outer member 100, past the distal end of the inner member 100, and toward the treatment site. The second inner member may be utilized to deliver a different agent, for example. Additionally, or alternatively, the outer member 100 may be moved to a different targeted treatment site in between use of the first and second inner members.

In some cases, the inner member may require more structural rigidity to move towards the treatment site. In these cases, the stylette 300 may be used. The stylette 300 provides greater stiffness to the inner member 200 to aid in guiding the inner member 200 closer to the treatment site. In other embodiments, the inner member 200 may be capable of reaching the targeted anatomy without the aid of the stylette 300. In these cases, the stylette 300 need not be inserted into the inner member 200. The inner member 200 may include a stopping element at the distal section configured to prevent the stylette's distal section from pushing distally past the inner member 200. The stopping element may be, for example, a notch, friction fit, or other appropriate means to stop the stylette's distal end from pushing past the inner member's distal end.

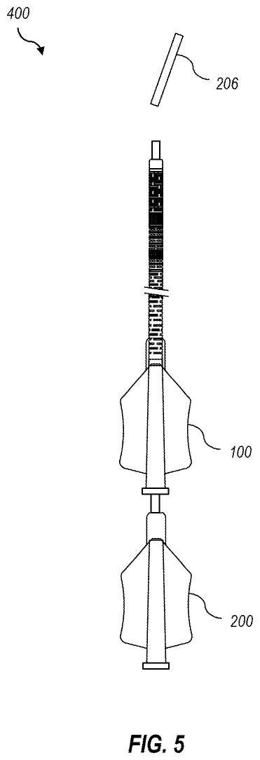

FIGS. 4 and 5 illustrate an example of the catheter system 400 including the inner member 200 having a selectively detachable distal section 206. In FIG. 5, the selectively detachable distal section 206 has been detached. An example of when this feature could be used is in the case where the inner member 200 is formed of a material that bonds to the embolic agent that is injected into the catheter system 400. In this example, if the distal section 206 gets stuck in a bolus of the adhesive after injecting the embolic agent, the distal section 206 may be detached. The detachable distal section 206 may be configured to detach due to perforation, groove, thinned section, partial break, an intentional weak spot, or other means appropriate to allow the distal section 206 to selectively detach when sufficient tension is applied. In other embodiments, the inner member 200 may omit a selectively detachable distal section 206. For example, if the inner member 200 is made of a material that does not bond to the embolic adhesive, the detachable distal section 206 may be unnecessary. In other instances, the inner member may be used to deliver some component other than an adhesive (e.g., medication), also making the detachable distal section 206 unnecessary.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A catheter system, comprising:
an outer member that includes a lumen extending therethrough;
an inner member including a distal section and a proximal section, the inner member being configured to fit inside the lumen of the outer member, wherein the inner member is translatable within the outer member to selectively extend beyond a distal end of the outer member; and
a stylette configured to fit within a lumen of the inner member, wherein the stylette has greater bending stiffness and/or columnar stiffness than the inner member.

2. The catheter system of claim 1, wherein the proximal section of the inner member has greater stiffness than the distal section of the inner member.

3. The catheter system of claim 1, wherein the stylette includes a distal and proximal section, wherein the distal section of the stylette is microfabricated to have greater flexibility than the proximal section.

4. The catheter system of claim 1, wherein the stylette has a radiopaque marker disposed at or near its distal end.

5. The catheter system of claim 1, wherein the stylette includes a hydrophilic coating.

6. The catheter system of claim 1, wherein a proximal end of the inner member includes a Luer connection.

7. The catheter system of claim 1, wherein the inner member is configured to avoid bonding to an adhesive passed therethrough.

8. The catheter system of claim 1, wherein the distal section of the inner member is selectively detachable from the proximal section of the inner member.

9. The catheter system of claim 8, wherein the distal section of the inner member includes a weak portion that enables selective detachment of the distal section from the proximal section under applied tension.

10. The catheter system of claim 1, wherein the outer member is formed of nitinol and/or a polymer.

11. The catheter system of claim 1, wherein at least a distal section of the outer member is microfabricated to include circumferentially extending rings and axially extending beams.

12. The catheter system of claim 11, wherein the outer member comprises a polymer disposed within spaces defined by the rings and beams.

13. The catheter system as in claim 1, wherein the inner member is formed from a plurality of polymers.

14. The catheter system of claim 13, wherein the plurality of polymers have varying stiffness and are arranged to provide a gradient stiffness that decreases toward a distal end of the inner member.

15. The catheter system of claim 1, wherein the inner member includes a stopping element configured to prevent the distal end of the stylette from passing distally beyond the stopping element.

16. A catheter system, comprising:
an outer member that includes a lumen extending therethrough;
an inner member including a distal section and a proximal section, the inner member being configured to fit inside the lumen of the outer member, wherein the inner member is translatable within the outer member to selectively extend beyond a distal end of the outer member, and wherein the proximal section of the inner member has greater stiffness than the distal section of the inner member; and
a stylette configured to fit within a lumen of the inner member, wherein the stylette has greater bending stiffness and/or columnar stiffness than the inner member, and wherein the distal section of the inner member is selectively detachable from the proximal section of the inner member.

17. The catheter system of claim 16, wherein the distal section of the inner member includes a weak portion that enables selective detachment of the distal section from the proximal section under applied tension.

18. A method for embolizing targeted anatomy, comprising:
providing the catheter system of claim 1;
guiding the outer member towards the targeted anatomy;
guiding the inner member to the targeted anatomy by translating the inner member within the outer member, wherein the distal end of the inner member is extended past the distal end of the outer member;
injecting an embolic agent toward the targeted anatomy; and
withdrawing the inner member from the outer member.

19. The method of claim 18, further comprising inserting an additional inner member into the outer member and injecting embolic agent through the additional inner member.

20. The method of claim 18, further comprising detaching the distal section of the inner member following injection of the embolic agent.
